(19) United States
(12) Patent Application Publication
(10) Pub. No.: US 2003/0211088 A1
(43) Pub. Date: Nov. 13, 2003

(54) Multipotent cell and cardiomyocyte cell populations, and routes to and uses of same

(76) Inventor: Loren J. Field, Indianapolis, IN (US)

Correspondence Address: Woodard Emhardt Naughton, Moriarty & McNett, Suite 3700, 111 Monument Circle, Indianapolis, IN, 46204, US

(21) Appl. No.: 10/220,205

(22) PCT Filed: Mar. 12, 2001
(86) PCT No.: PCT/US01/07674

(51) Int. Cl. A61K 48/00; C12N 5/08

(52) U.S. Cl. 424/93.21; 435/366

(57) ABSTRACT

Described are conduction cardiomyocyte-enriched cellular populations, and methods and materials for obtaining the same. The populations may be used to engraft mammalian myocardial tissue, for example to provide biological pacemakers. Also described are restorative cellular myocardial tissue, for example to provide biological pacemakers. Also described are restorative cellular myocardial grafts for improving the contractile function of injured segments of myocardium, and articles adapted for heart implantation (e.g. conductive pacemaker leads), which includes coatings of viable cardiomyocytes and optionally a carrier for the cardiomyocytes.

### REFERENCE TO RELATED APPLICATION

This application claims the benefit of U.S. Provisional Application Serial No. 60/188,507 filed Mar. 10, 2000, which is hereby incorporated herein by reference in its entirety.

### BACKGROUND OF THE INVENTION

The present invention relates generally to the field of cardiology, and in particular to cardiomyocyte cell populations, and methods for obtaining and using the same.

As further background, the conduction system of the heart determines heart rate in response to signals from the nervous system as well as chemical signals delivered from other organs. The conduction system provides impulses to the myocardium in a coordinated fashion, which impulses facilitate the coordinative action of various sections of the heart to pump blood. This conduction system is supported by the regular generation of a depolarization wave which causes contraction of the myocardium. The coordination of the location and frequency of the depolarization wave is critical in rendering the heart responsive to its own needs as well as other needs of the body.

A variety of diseases or disorders are known which impair the conduction system. Thus, the concept of “pacing” the heart has been developed, in which a stimulus is supplied to the heart to generate a depolarization wave and attendant contraction of the heart. For example, it is common today to utilize an electromechanical pacemaker to apply regular stimuli to the heart to facilitate a regular heartbeat in patients lacking sufficient natural heart pacing function. In a typical case, an electrode of the pacemaker is implanted in myocardial tissue at the apex of the right ventricle, and the remaining pacemaker components are implanted under the patient's skin, typically near the chest.

The use of such electromechanical pacemakers has presented several problems. For example, batteries of such pacemakers are usually exhausted after several years, and when this happens a new pulse generator must be implanted. Further complications can arise from the need to implant the electrode in the heart. For instance, it has been found that an area of fibrosis often develops around the embedded electrode. This fibrosis inhibits communication of the lead with healthy myocardial tissue, and increases the size of the signal necessary to pace the heart. Consequently, the batteries of the pulse generator are more quickly exhausted, and must be more frequently replaced.

Additional disadvantages in utilizing electromechanical pacemakers relate to their lack of responsiveness to changes in oxygen demand by organs of the body. Thus, limitations are imposed as to the physical activities of the user. While some exercise and other responsive pacemakers have been and are being developed, a satisfactory solution to this problem has yet to be attained.

Given the difficulties and shortcomings of electromechanical pacemakers, the concept of providing a “biological pacemaker” has been entertained for some time in the field of cardiology. As envisioned, such a biological pacemaker would involve a graft of live tissue on the heart which would provide regular impulses to trigger depolarization waves to pace the heart. Several obstacles have yet to be overcome in prior work to realize the goal of a biological pacemaker. For example, a suitable and effective tissue or cell population for engraftment has yet to be identified. Moreover, issues pertaining to the longevity and stability of pacemaker grafts need to be addressed.

As an example of work in the field of biological pacemakers, U.S. Pat. No. 5,103,821 describes an attempt to formulate a protocol for a biological pacemaker. In particular, the '821 patent proposes to harvest sino-atrial node cells from an area of the heart in the right atrium where the cells are located. Removal of these cells is accomplished by catheters which include small biopsy devices which remove a small quantity of cells but do not penetrate the right atrium. The next step in the '821 process is to culture the harvested sino-atrial node cells to generate a larger quantity of cells. The cultured cells are then introduced into the right ventricle of the heart using an implant catheter, in particular at the apex of the right ventricle, a location which experience with electromechanical pacemakers has demonstrated is advantageous for initiating the polarization waves. The '821 patent proposes that this process will provide the patient with a biological pacemaker which functions almost identically to the natural pacing of the heart.

A difficulty with the '821 proposal is that experience has shown that the culture of adult cardiomyocytes is not a straightforward endeavor, and in fact to date the applicant knows of no suitable methods for culturing adult cardiomyocytes as would enable the conduct of a procedure as proposed in the '821 patent.

The conduction system of the heart is known to involve at least three cell types, including sino-atrial node cells, atrial-ventricular node cells, and Purkinje cells. The sino-atrial node is the primary heart pacemaker and is located at the junction of the superior caval vein and the right atrium. Immunohistochemical studies demonstrate that sino-atrial node cells can clearly be differentiated from atrial myocytes, each with its own characteristic phenotype. P. W. Oosthoek et al., Circulation Research, Vol. 73, No. 3, pp. 473-481 (1993). The atrial-ventricular node is composed of small myocytes embedded in connective tissue. The atrial-ventricular node terminates in finger-like myocytes that make contact with myocytes of the atrial-ventricular bundle, and Purkinje cells form a network of Purkinje fibers in the myocardium which deliver excitation current to the working myocardium. P. W. Oosthoek et al., Circulation Research, Vol. 73, No. 3, pp. 482-491 (1993). In addition to such immunohistochemical studies, research into the development of the heart including its conduction system has involved in vitro differentiation of embryonic stem cells. For example, it has been observed that embryonic stem cells differentiate into cells with properties of the sinus node, atrium, and ventricle. V. A. Maltsev et al., Circulation Research, Vol. 75, No. 2, pp 233-244 (August 1994); A. M. Wobus et al., Annals New York Academy of Sciences 752, pp 460-469 (March 1995).

Another area of cardiologic research concerns the diagnosis and treatment of damaged myocardial tissue to restore contractile function to the heart. Several conditions injure tissue composed of working atrial or ventricular cardiomyocytes. Such conditions include, for instance, tissue necrosis stemming from ischemic conditions such as coronary blockage myocardial infarction or the like. While efforts have been made to develop satisfactory therapies to re-establish contractile activity lost due to such injuries, they have been hindered by the generally non-regenerative character of the patient's existing cardiomyocytes.

In light of the foregoing background, there exist needs for improving, supplementing or avoiding the use of electromechanical pacemakers. In addition, there exist needs for improved methods for grafting myocardial tissue to provide for pacing or restoration of contractile function. Further needs exist for cardiomyocyte cell populations useful in implementing these grafts as well as for in vitro studies of the heart and pharmacological and toxicological screening. The present invention addresses these needs.

### SUMMARY OF THE INVENTION

In one of its preferred embodiments, the invention provides a cellular population comprising cardiomyocytes and which is enriched in conduction cardiomyocytes relative to working cardiomyocytes. Preferably, of the cardiomyocytes in the population, at least 50% are conduction cells, e.g. sino-atrial and/or atrial-ventricular node cells. Such a population may be obtained by a method of the invention, which comprises (i) providing a multipotent cell such as an embryonic stem cell which differentiates to cardiomyocytes, the multipotent cell including a conduction cardiomyocyte selection marker which enables selection of conduction cardiomyocytes from other cardiomyocytes; (ii) causing the multipotent cell to differentiate; and, (iii) selecting the conduction cardiomyocytes based on the selection marker. In this method, the selection marker can enable a positive or a negative selection protocol, with illustrative selection markers including antibiotic resistance genes and the HSV thymidine kinase gene. These elements can be combined to achieve a single-step selection protocol, in which the desired conduction cardiomyocyte population is obtained in a single selection, or a multi-step selection protocol, e.g. involving a stem cell with introduced DNA enabling an initial selection of a general cardiomyocyte population including conduction cardiomyocytes and working cardiomyocytes, and also enabling a subsequent selection of a conduction cardiomyocyte-enriched population from the general cardiomyocyte population.

The present invention also provides a transgenic multipotent (also known as pluripotent) cell such as an embryonic stem cell, which includes DNA having a selection gene fused to a promoter, wherein the DNA enables selection of a cell population enriched in conduction cardiomyocytes relative to working cardiomyocytes. Such stem cells may be constructed by transfecting an embryonic stem cell with a vector including the DNA. Such methods and vectors also constitute embodiments of the present invention.

The invention also concerns a method for pacing a heart in a mammal, which includes establishing in the heart a stable cellular graft of fetal cardiomyocytes including node cells, wherein the node cells pace the heart.

In another aspect, the present invention provides an animal model useful for cardiologic study. In particular, the invention provides a non-human mammal having a heart and a stable graft of fetal cardiomyocytes in the heart, wherein the cardiomyocytes are enriched in node cells relative to other cardiomyocytes.

Another aspect of the invention concerns a method for restoring function to an injured regional segment of the myocardium of a mammal. The method includes implanting viable cardiomyocytes in the regional segment.

The invention also concerns a device which includes a substrate adapted for in vivo implantation into myocardial tissue, and viable cardiomyocytes coated on the substrate. A preferred device is a conductive lead, for example a pacemaker lead. The device may also be an elongate filament, such as a suture. The substrate may be coated directly with the cardiomyocytes, or the device may include a carrier coated on the substrate, for instance a gel, carrying the viable cardiomyocytes.

In its several embodiments, the present invention provides cardiomyocyte and multipotent cell populations, and methods for obtaining and using the same. The invention also provides related vectors, animal models and engraftment procedures for heart pacing and restoration of contractile activity, as well as improved articles for heart implantation coated with viable cardiomyocytes. The invention thereby provides improvements both in the implementation of conventional electromechanical devices, and in avoiding or supplementing the function of such electromechanical devices.

Additional embodiments, objects and features of the invention will be apparent from the description which follows.

### DESCRIPTION OF THE PREFERRED EMBODIMENTS

For the purposes of promoting an understanding of the principles of the invention, reference will now be made to certain embodiments thereof and specific language will be used to describe the same. It will nevertheless be understood that no limitation of the scope of the invention is thereby intended, such alterations, modifications and further applications of the principles of the invention as illustrated herein being contemplated as would normally occur to one skilled in the art to which the invention relates.

### Multipotent Cell and Cardiomyocyte Cell Populations

As disclosed above, the present invention features enriched populations of fetal conduction cardiomyocytes and methods and materials for obtaining and using the same. In this regard, the fetal conduction cardiomyocytes utilized in the present invention will have the electrophysiologic properties consistent with pacemaker cells which act to establish depolarization waves in the heart. Thus, the conduction cardiomyocytes will generally demonstrate automaticity (i.e. spontaneous contractile activity) such as that displayed by sino-atrial (SA) node cells and/or atrial-ventricular (AV) node cells.

Substantially enriched populations of conduction cardiomyocytes can be obtained for use in the invention by the differentiation of a genetically engineered multipotent cells, illustratively embryonic stem (ES) cells, somatic stem cells, or other multipotent cells, having means for selecting conduction cardiomyocytes from other cardiomyocytes to which the stem cells differentiate, such as working atrial and ventricular cardiomyocytes. Preferred embryonic stem or other multipotent cells of the invention will thus include introduced DNA encoding a selection marker fused to a promoter, which provides for (positive or negative) selection of conduction cardiomyocytes from other cardiomyocytes. In this regard, as is known in the art, in a positive selection scheme, a selection marker conferring increased survivability to a selection pressure is fused to a promoter which causes expression of the selection marker in the cell type to be selected, but not in cell types to be eliminated. Thus, illustratively, to obtain a conduction cardiomyocyte-enriched population in accordance with the invention, a positive selection gene, such as a gene conferring resistance to an antibiotic or other lethal agent, is fused to a promoter which causes expression of the gene in conduction cardiomyocytes but not in other cardiomyocyte types. Subjecting a differentiated population containing the conduction and other cardiomyocytes to the corresponding selection pressure, e.g. the antibiotic, will then provide a selected population which is enriched in viable conduction cardiomyocytes relative to the initial population.

On the other hand, in a negative selection scheme, a selection gene is used which decreases survivability of the cell in the presence of a selection pressure. This selection gene is fused to a promoter causes its expression in cell types other than those to be selected, but not in the cells to be selected. The mixed population is then subjected to the selection pressure to provide a cell population which is enriched in the selected cell types relative to the initial population. As an example, the HSV thymidine kinase (HSV-TK) gene is commonly used in negative selection schemes. This gene confers susceptibility to the agent gancyclovir in cells which express it. Thus, to obtain a population that is enriched in node cell cardiomyocytes, an embryonic stem cell or other multipotent cell can be provided carrying a negative selection gene such as the HSV thymidine kinase gene fused to a promoter which is inactive in conduction cardiomyocytes but active in other cardiomyocytes, for example the connexin43 promoter (Oosthoek et al., Circulation Research, Vol. 73, No. 3, pp. 473-481 (1993); Oosthoek et al., Circulation Research, Vol. 73, No. 3, pp. 482-491 (1993)). Differentiation of the stem cell will result in mixed population containing conduction cardiomyocytes and other cardiomyocytes, e.g. working cardiomyocytes. The negative selection gene, e.g. the HSV thymidine kinase gene, will not be expressed in the conduction cardiomyocytes, which will thus not be susceptible to the negative selection agent, e.g. gancyclovir. The negative selection gene will be expressed in other cardiomyocytes (e.g. working cardiomyocytes), which will thus be susceptible to the negative selection agent, e.g. gancyclovir. Incubation of the mixed cardiomyocyte population containing the conduction and other cardiomyocytes in the presence of the negative selection agent will kill the other cardiomyocytes, but not the conduction cardiomyocytes. A conduction cardiomyocyte-enriched population is thereby obtained.

Selection methods of the invention may include one selection step or multiple selection steps, so long as at least one selection step enriches the population in conduction cardiomyocytes relative to other cardiomyocytes. An illustrative one-step selection may be provided with an embryonic stem cell containing a positive selection gene (e.g. an antibiotic resistance gene) fused to a promoter which is specific to conduction cardiomyoytes in the context of the selection protocol (i.e. the promoter is active essentially only in conduction cardiomyocytes and not any other cell type differentiated from the embryonic stem cell at the time of selection). This stem cell is differentiated to provide a population including the conduction cardiomyocytes and other cells, which population is subjected to the positive selection pressure (e.g. the corresponding antibiotic agent) to kill the other cell types, leaving a selected, viable conduction cardiomyocyte-enriched population. Illustrative positive selection genes which may be used for these purposes include the neomycin and hygromycin resistance genes. Illustrative promoter candidates for these purposes include promoters specific to or enhanced in conduction cardiomyocytes including, for instance the minK promoter.

As an illustrative multiple-step selection procedure, an embryonic stem or other multipotent cell may be provided having means for selecting a first population differentiated from the stem cell, and for selecting a second population from the first population. The embryonic stem or other multipotent cell may, for instance, carry means for selection of a general cardiomyocyte population including conduction and other cardiomyocyte types, and then for selecting a conduction cardiomyocyte-enriched population from the general cardiomyocyte population. As an example, the multipotent cell may carry a positive selection gene such as an antibiotic resistance gene fused to a promoter active in cardiomyocytes but not other cell types. Suitable such promoters include for instance the beta-MHC promoter, and can be used to prepare highly-purified fetal cardiomyocyte populations as reported in WO 95/14079 (May 26, 1995). The multipotent cell can also be engineered to contain a negative selection gene fused to a promoter which is inactive in conduction cardiomyocytes but active in other cardiomyocytes. Suitable negative selection genes include for instance the HSV-TK gene. Suitable candidate promoters for these purposes include for example the connexin43 promoter. Such a stem cell can then be differentiated to provide an initial cell population containing cardiomyocytes and other cell types, and the positive selection pressure applied to select a general cardiomyocyte population. Thereafter, the negative selection pressure can be applied to the general cardiomyocyte population to select the conduction cardiomyocyte-enriched population.

The minK promoter may be used to facilitate obtaining node cell-enriched populations. It has been reported that minK promoter is restricted to or at least enhanced in the conduction system of the heart. Kupershmit et al., Circulation Research, Vol. 84, No. 2, pp. 146-152 (February 1999). This promoter is fused to a selection gene such as the amino glycoside phosphotransferase fusion gene to permit G418 selection of differentiated cultures of embryonic stem or other multipotent cells as generally described above. After establishing a clonal undifferentiated embryonic stem or other multipotent cell line, the cells are differentiated in vitro. G418 selection is imposed once beating cells (i.e. cardiomyocytes) are observed. Identification of cells with molecular and electrophysiologic attributes consistent with conduction cardiomyocytes but not working ventricular and atrial cardiomyocytes, establishes that conduction cardiomyocytes are selected by this in vitro differentiation process and enables a determination of the extent of enrichment in conduction cardiomyocytes. For example, conduction cardiomyocytes and working ventricular and atrial cardiomyocytes can be discerned from one another by differing action potential profiles and/or patterns of connexin expression.

In a still further method for obtaining node cell-enriched populations, a multipotent cell such as an embryonic stem cell is provided including a alpha-MHC promoter fused to a neomycin resistance gene, and also including a fusion gene comprised of the connexin43 promoter fused to the (HSV-TK) gene. After in vitro differentiation of the multipotent cells, cardiomyocytes in general are selected by incubation with G418. Afterward, a second round of selection with gangcyclovir is initiated. Cells expressing the HSV-TK gene will incorporate the gangcyclovir and die, whereas cells not expressing the HSV-TK gene will survive. Because the connexin43 promoter is active in the ventricular and atrial working cardiomyocytes, these cells will die. In contrast, the conduction cardiomyocytes do not express connexin43, will not express the HSV-TK gene and thus will survive gangcyclovir selection. Again, the extent of conduction cardiomyocyte enrichment in the selected cultures can be ascertained using molecular and electrophysiologic analyses.

### Cellular Engraftment for Biological Pacing

Multipotent cell-derived conduction cardiomyocyte-enriched populations obtained as described above or fetal cardiomyocytes can be used for example to generate biologic pacemakers in living mammals. Generally speaking, this involves the implantation of the multipotent cell-derived or fetal conduction cell-containing population into the heart tissue of a mammal (including human patients) in need of heart pacing, wherein the implanted population paces the heart.

In this regard, a mass of SA node cells located on the right atrium is responsible for pacing the heart in a normally-functioning heart. This mass is responsible for generating an initial depolarization wave which ultimately results in the contraction of the entire heart muscle and in particular the heart muscle forming the wall of the right ventricle and the wall of the left ventricle. In a typical cycle, the SA node cells initiate such a depolarization wave which spreads through the muscle tissue of the right atrium until the wave front arrives at another small knot of cells in the right atrium identified as the atrial ventricular (AV) node. The AV node is located low in the rear wall of the right atrium and passes downwardly within a wall which separates the right ventricle from the left ventricle. Long fibers extending into this separating wall, similar to nerve cells, form a bundle of fibers known as the Bundle of His and extend from the AV node along the length of the separating wall and into all parts of the ventricular myocardium.

Proper function of the heart is dependent upon an depolarization wave radiating from the SA node, through the right atrial and along the Bundle of His where the depolarization wave stimulates the myocardium of the left and right ventricles low in the heart to ensure that the depolarization or contraction of the muscle tissue progresses in a wave pattern which will produce a pumping action in the left and right ventricles.

Heart disease can result in impaired function of the SA node or can damage or interrupt the atrium surrounding the AV node, so that depolarization does not reach the AV node. This interrupts the depolarization wave to the left and right ventricles. In other impairments, fractures may occur in the Bundle of His or branches therefrom with the result that the depolarization wave generated by the SA node does not reach the right and left ventricles in a uniform and efficient fashion. In still other cases, complete heart blockage of the depolarization wave can occur.

In the present invention, the ES-derived or fetal conduction cells may be implanted in the heart in order to restore or improve the function of the heart in terms of pacing. To accomplish this, the conduction cells may be implanted within the native SA node or AV node structures, or elsewhere in the conduction system, to supplement the functions of these structures. Alternatively, a pacing graft of the conduction cells may be located in a separate region of the heart. For example, the pacing graft may be located in regions found to be effective for implantation of the lead of a conventional electromechanical pacemaker. Illustratively, this may include a location in the right ventricle of the heart, especially at or near the extreme apex of the right ventricle. Such location has been found with electromechanical pacemakers to provide an effective site for stimulation to achieve efficient contractile activity of the heart for pumping blood. This or other locations of the right ventricle are selected so as to generate a depolarization wave from the fetal or ES-cell-derived conduction cell graft which radiates in a uniform and orderly progression along the walls of the right and left ventricles. The engraftment procedure may involve catheterization of the heart so as to implant the cells at or near the internal surfaces of the heart. Alternatively, the cells may be engrafted on the exterior surface of the heart in an open or potentially laproscopic procedure. Depending on the particular situation, it may be necessary to destroy the function of the native conduction system, e.g. by ablating or otherwise inactivating the SA node. However, it is envisioned that this step should be avoided where possible.

A number of injections or implantations of cells may be necessary to provide pacemaker activity of the graft in larger mammals such as humans. Anticipated total graft sizes in humans are from about 10⁴ to about 10⁷ cells or more. These established, viable grafted cells can be provided by one or multiple cellular implantations, e.g. by implanting up to about 10⁸ or more cells at a time. In one preferred mode, the graft is established by multiple implantations (e.g. injections) of up to about 250,000 cells at a time. If necessary after a preliminary operation, one or more additional operations can be conducted to add to the size of the graft to optimize its biological pacing or other activity.

In addition to the conduction cardiomyocyte engraftment procedures described above, genetically-engineered cells, including the engrafted cardiomyocytes and/or other cells, can be used to promote neo-innervation of the graft. In particular, neo-innervation in a pacemaker graft can be achieved by targeting the expression of growth factors which promote neo-innervation, for example nerve growth factor (NGF) (see, e.g., U.S. Pat. Nos. 5,082,774; 5,169,762; 5,272,063; and 5,288,622). In illustrative experimental, an NGF expression cassette is introduced into skeletal myoblasts (C2C12 cells) and/or fibroblasts, stably transformed cell lines are established, and the capacity to produce biologically active NGF is ascertained in an in vitro neurite outgrowth assay. To monitor the degree of neo-innervation, the genetically-modified cells are engrafted into an adult syngeneic host. At three weeks post-engraftment, the animals are sacrificed, and harvested and processed for immunohistologic analyses. NGF fusion gene expression is monitored by in situ and anti-NGF immunohistologic analyses. The extent of neo-innervation is assessed using immunologic markers for both sympathetic and parasympathetic neurons, as well as by histologic examination and measurement of cardiac catecholamine content. Retrograde neuron labeling experiments are conducted with working heart preparations in vitro to track the anatomic origins of the nascent neurons within the heart. In all instances, corresponding grafts lacking the NGF transgene are employed as negative controls.

Thus, in this and similar engraftment techniques of the invention, conduction cardiomyocyte-enriched populations can themselves deliver proteins such as nerve growth factor, or can be co-engrafted with other cells delivering such proteins, to optimize the establishment and performance of the biological pacemaker graft. Once again, immunohistologic analyses are used to monitor the extent of neo-innervation. For in vivo analysis, neo-innervation may be monitored by non-invasive techniques, for instance by PET scanning utilizing a suitable imaging agent such as 11-C-hydroxy epheddrine. Negative controls in such experimentation include grafts lacking the engrafted cell type (e.g. myoblasts or fibroblasts), as well as grafts with the engrafted cell type but lacking the NGF transgene. Once the extent of anatomic neo-innervation is established, additional experiments may be performed to determine the extent of functional innervation. For example, retrograde labeling techniques can be used to determine the coupling of the pacemaker graft to the cardiac ganglia. Subsequent physiological experiments can be used to confirm that the pacemaker graft is subject to autonomic regulation in instances where AV node activity is blocked pharmacologically and/or physically.

### Cellular Engraftment for Restoration of Contractility

The present invention also concerns cellular engraftment procedures which are used to restore function to regions of the myocardium which have suffered injury resultant, for example, of regional tissue injury stemming from ischemia, apoxia, hypoxia or the like. For example, the blockage or restriction of a coronary artery, such as by a blood clot, can lead to injury of a particular area of the heart served by the artery due to inadequate blood supply. As a result, the area of injury may involve impairment or death of the myocardial tissue in the region. Such conditions cause a decrease in the contractile function of the heart and in the efficiency with which it pumps blood. In accordance with the present invention, after conventional fibrinolitic or other therapy for such injuries, an engraftment procedure using cardiomyocytes or other suitable cells may be conducted to restore function to the region of injured myocardium.

Preferred restorative grafts of the invention will be located at or adjacent to the region of injured myocardium, and will desirably be introduced as soon as possible after the infarct or other injury, during active granulation tissue formation but prior to scarring and myocardial thinning. Preferred grafts will include cardiomyocyte cells which are physically and electronically coupled to the viable native myocardium adjacent to the injured myocardium. Such coupling can be observed, for example, by the organization of the engrafted cells and the formation of nascent junctional complexes both between engrafted cells themselves and between engrafted cells and the native cardiomyocytes. Several implantations of cardiomyocytes may be necessary to provide the restorative properties to the graft. These may be provided, for example, by one or more implantations of cells, with each implantation having up to about 10⁸ cells or more. In a preferred method, the graft is established by multiple implantations (e.g. injections) of up to about 250,000 cells at a time to achieve a graft size of about 10⁴ to 10⁷ cells or more. In preferred grafts, at least one implantation of cells will be made such that the engrafted cells contact viable-myocardial tissue for instance on the perimeter of the injured region of myocardium. Where larger injured regions are involved, multiple injections or implantations of cells may be made around the periphery of the injured region so as to substantially surround the injured region with engrafted, viable cells. Furthermore, in subsequent implantations conducted in the same operative procedure or in follow-up procedures, cells from a prior engraftment may be used as the newly established periphery, and grafts may be constructed so as to provide viable, coupled cardiomyocytes substantially into or substantially throughout the injured myocardial region, so as to provide the improved functionality to the region. After one or multiple procedures for restorative engraftment, the patient can be monitored for improvement in contractile function of the heart and the loss or decrease in functional artifacts caused by the injured region of myocardium. Such monitoring can be conducted by signal processing methodologies which are known and used in the detection and localization of ischemic or other injured myocardium.

The capacity to restore function to injured myocardium has been demonstrated by applicant in open chest procedures on dystrophic dogs. In particular, functional restoration of an injured myocardial region has been observed in tests of segment length shortening using implanted pairs of sonomicrometer crystals (see Example 4 below). In dogs receiving cellular engraftment of fetal cardiomyocytes by injection between the sonomicrometer crystals, a 5% increase in contractility was observed in response to subsequent dobutamine challenge. On the other hand, in dogs not receiving cellular engraftment and instead receiving only an injection of saline between the crystals, as a result of the injured myocardium in the injected segment, the segment did not respond with an increase in contractility during dobutamine challenge.

### Cellular Interface for Myocardial Implants

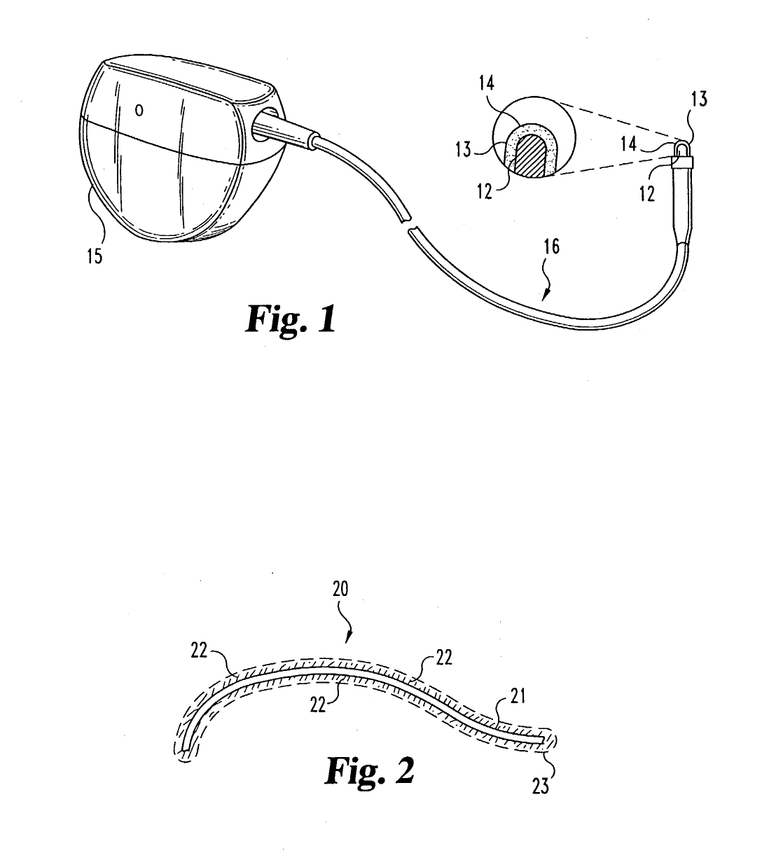

Another aspect of the invention concerns the use of viable cardiomyocytes to modify articles adapted for in vivo implantation into the heart. For instance, in one aspect, the invention provides means to generate a cellular interface between a conductive lead and the host myocardium, e.g. for an electromechanical pacemaker, defibrillation device, or the like. As is known, traditional pacemakers activate ventricular depolarization through a bipolar lead which field stimulates a region of the endocardial surface in the apex of the ventricular. Because the electrical signal is dissipated through 360° C., and because there is frequently fibrosis between the lead and the excitable myocardium, a significant amount of energy is needed to trigger cardiac depolarization. In the present invention, a cellular interface is constructed between the conductive lead and the excitable myocardium. In particular, in a preferred form, the invention provides a conductive lead which includes a carrier impregnated with viable cardiomyocytes. The carrier can for example be a gel such as a collagen gel. The lead can by any lead known for heart implantation, including for instance straight, helicle, barbed and other lead types. FIG. 1 shows one such lead. In particular, shown is device 11 adapted for fixed implantation into the heart tissue. Device 11 includes conductive lead 12 coated with a carrier layer 13. Carrier layer 13 can be formed of any suitable carrier material which maintains the viability of the cardiomyocytes. Preferred carriers will be gels, for example collagen gels. Contained in carrier layer are viable cardiomyocytes 14. The conductive lead is electrically connected to an electromechanical device 15, e.g. a pulse generator of a pacemaker, via wire 16.

In use, the tip of the lead 12 including the carrier 13 and cardiomyocytes is inserted into the myocardium, facilitating coupling between the viable cardiomyocytes 14 and the excitable myocardium, and the electromechanical device 15 is conventionally implanted in the patient. Because the pacemaker discharge from the pulse generator 15 does not have to traverse a region of fibrosis (or has to traverse only a smaller region of fibrosis), and because the discharge is not dissipated through a 360° C. area, stimulation of the interface cardiomyocytes 14 can be accomplished with lower energy utilization. Excitation of the host myocardium is actuated through nascent junctional complexes formed between the host and interface cardiomyocytes (preferably fetal cardiomyocytes.). The use of such cellular interfaces between pacemaker leads can for example reduce the energy needed to stimulate ventricular depolarization as compared to the methods currently used for field stimulation. This in turn can extend the useful battery life in current systems.

Referring now to FIG. 2, shown is another article of the invention 20 which includes an elongate filament 21, for example a suture, having viable cardiomyocytes 22 coated thereon. Article 20 optionally may also include a carrier layer 23 (shown in phantom) to facilitate adherence of the cardiomyocytes 22 to the filament 21; however, it has been found that cardiomyoytes have a natural tendency to adhere directly to surfaces, even without the carrier. Article 20 is useful, e.g., in the in vitro study of the electrophysiologic properties of cardiomyocytes 22, including their ability to transmit a signal along the length of the filament 21. These studies can be conducted in the context of research and/or in the screening of agents for their toxicologic or pharmacologic effects on the cardiomyocytes 22 and their ability to transmit electronic signals consistent with participation in or initiation of a depolarization wave. Article 21 may also be used in vivo by implanting article 21 into myocardial tissue to similarly study signal transmission and/or to modify the existing pattern of initiation and/or propagation of depolarization waves in the heart. For example, article 20 may be implanted into the myocardium so as to span between two regions, and thus provide a filamentous cellular graft therebetween. Such graft may then serve to modify signal transmission between the two regions, participating for instance in the enhancement of the propagation of a depolarization wave between the two regions to improve the efficiency of heart pumping. For example, in some situations, the cell-populated filament may be implanted to span from the SA nodal region to the left ventricular wall, e.g. in the treatment of dilated cardiomyopathy.

In order to promote a further understanding of the present invention and its advantages, the following specific examples are provided. It will be understood that these examples are illustrative and not limiting of the invention.

### EXAMPLE 1

### Selection of ES Derived Pacemaker Cells

This Example details selection protocols for obtaining cell populations enriched in cardiomyocyte sub-lineages, including node cells. It has been shown that a cardiac lineage-specific promoter (i.e. the alpha-cardiac myosin heavy chain promoter) fused to a drug resistance gene (i.e. a cDNA encoding aminoglycoside phosphotransferase) can be used to select essentially pure cultures of cardiac myocytes in differentiating cultures of ES cells (see U.S. Pat. No. 5,602,301 and WO 95/14079, each hereby incorporated by reference). Molecular and electrophysiological analyses have indicated that cells with attributes similar to working atrial and ventricular and conduction cells are present in the G418-selected cultures taught in WO 95/14079. A number of protocols for selecting cardiac sub-lineages from such differentiated are given below.

A. Isolation of ES-Derived Ventricular Cardiomyocytes.

A fusion gene comprising the MLC2v promoter driving expression of a cDNA encoding aminoglycoside phosphotransferase is constructed. The fusion gene also contains a phosphoglycerate kinase (pGK)-hygromycin cassette. Undifferentiated ES cells are transfected with the MLC2v-neo/pGK-hygro fusion gene. Transfected cells are initially selected and cloned by virtue of their resistance to hygromycin. The resultant clonal lines are subsequently differentiated in vitro, and subjected to G418 selection when cardiomyocyte differentiation is apparent (i.e. once beating cells are detected). The resulting population of selected cells is examined for cardiac- and ventricular-specific markers to ascertain he degree of lineage-specificity in the selected cell population. Markers examined include ANF, MLC2a, MLC2v, a-MHC, β-MHC and cTnT. Identification of cells with attributes consistent with ventricular cardiomyocytes, but not atrial and conduction cardiomyocytes, or relatively fewer atrial and conduction cardiomyocytes establishes that a cardiomyocyte cell population enriched in ventricular cardiomyocytes can be selected by this in vitro differentiation process.

B. Isolation of ES-Derived Pacemaker Cells Using the MinK Promoter.

A mouse minK-βGAL fusion gene is constructed. Transgenic mice are generated with this construct using conventional methodology to ascertain the cell-type specificity of cardiac minK promoter. Mice resulting from embryos injected with the minK-βGAL construct are screened to identify transgenic founders. These animals are bred, and the pattern of βGAL activity is monitored in fetal, neonatal and adult transgenic offspring. These experiments establish the temporal cardiac-specific pattern of expression. In the event that minK expression is restricted or enhanced in the conduction system, this promoter is used too for selection in ES-derived cardiomyocytes. Once again, the promoter is incorporated into an aminoglycoside phosphotransferase fusion gene to permit G418 selection of differentiating cultures of ES cells. Molecular and electrophysiological criteria are used to establish the degree of conduction system selection.

C. Isolation of ES-Derived Pacemaker Cells Using a Double Selection Scheme.

In this protocol, a transgene comprised of an α-MHC promoter fused to a neomycin resistance gene is used to select a mixed population of cardiomyocytes of atrial, ventricular and conduction system origin This system is expanded to a double selection scheme to eliminate the non-conduction system cells. In particular, the connexin43 promoter is used, which is active in working ventricular and atrial cardiomyocytes, but not in node cells. A fusion gene comprised of the connekin43 promoter and the Herpes Simplex Virus thymidine kinase gene (HSV-TK) is introduced into ES cells carrying the MCH-neo^{r} construct. After in vitro differentiation, cardiomyocytes are selected by incubation with G418. A second round of selection with gangcyclovir is then initiated. Cells expressing the HSV-TK gene incorporate the gangcyclovir and die, whereas cells not expressing the HSV-TK gene survive. As the connexin43 promoter is active in the ventricular and atrial working cardiomyocytes, these cells will die. In contrast, the node cells do not express connexin43, will not express the HSV-TK gene and thus survive gangcyclovir selection. Molecular and electrophysiologic analyses are performed to ascertain the extent of conduction cell enrichment in these selected cell cultures.

D. Isolation of ES-Derived Pacemaker Cells Using the Connexin40 Promoter.

Studies have indicated that connexin 40 is expressed in the Purkinje cells but not in working cardiomyocytes (Oosthoek, P. W., S. Viragh, W. H. Lamers, and A. F. Moorman. Immunohistochemical delineation of the conduction system. II: The atrioventricular node and Purkinje fibers. Circulation Research. 73: 482-491, 1993; Oosthoek, P. W., S. Viragh, A. E. Mayen, M. J. van Kempen, W. H. Lamers, and A. F. Moorman. Immunohistochemical delineation of the conduction system. I: The sinoatrial node. Circulation Research. 73: 473-481, 1993). Transgenes are generated which utilize the connexin 40 promoter (Seul, K. H., P. N. Tadros, and E. C. Beyer. Mouse connexin40: gene structure and promoter analysis. Genomics Nov. 15, 1997 46: 120-126, 1998) to drive expression of a neomycin resistance expression cassette. The construct is built into a pGH-hygro^{r} vector backbone. ES cells are transfected with the Connexin 40-neo^{r}/pGK-hygromycin transgene. 24 hrs later, the cells are subjected to hygromycin selection. When the hygromycin-resistant clones are sufficiently amplified, the cultures are induced to differentiate. Once beating cells are detected (8 days post-induction), the cultures are subjected to G418 selection.

Once selected cultures are in hand, the degree of cardiomyocyte enrichment is ascertained. In preliminary experiments, G418-selected cultures are digested with trypsin and replated onto collagen coated chamber slides at a density suitable for immune cytologic analysis of individual cells. 24 hours post plating, the cells are screened for sarcomeric myosin cardiomyocyte enrichment when using atrial-restricted promoters. Once the extent of cardiomyocyte enrichment is established, immune cytologic analyses is used with connexin 40- and 43-specific antibodies to ascertain the relative content of Purkinje cells. Northern blot analyses with connexin 43 and 40 probes are also be performed. RNA is prepared from G418-selected cardiomyocytes expressing either the MHC-neo^{r} or the Connexin 40-neo^{r} transgenes. If the selection protocol works as expected, Connexin 40 should be the predominant species in the Connexin 40-neo^{r} cultures and Connexin 43 in the MHC-neo^{r} cultures.

E. Alternative Approach to Cardiomyocyte Isolation

Using an alternative approach, one can take advantage of the recent studies by Takashi Mikawa and co-workers which indicate that, during chick embryogenesis, development of Purkinje fibers arises by transdifferentiation of working ventricular cardiomyocytes (see Gourdie, R. G., Y. Wei, D. Kim, S. C. Klatt, and T. Mikawa. Endothelin-induced conversion of embryonic heart muscle cells into impulse-conducting Purkinje fibers. PNAS Jun. 9, 1998. 95: 68-6815-6818, 1998). These studies demonstrated that endothelin-1 (ET-1, a potent vasoconstrictor secreted by vascular endothelial cells) promotes this transdifferentiation process. Accordingly cultures of ES cells carrying the MHC-neo^{r} transgene are treated with ET-1 during the differentiation and selection protocol to determine if a similar transdifferentiation process can be promoted in mammalian cardiomyocytes. The capacity of early murine fetal cardiomyocytes to transdifferentiate into Purkinje cells in vitro is also tested. In both cases, the cultures are exposed to ET-1 (initial experiments entail simple dose/response studies). After treatment, RNA is prepared from the cultures and the relative working cardiomyocyte vs. Purkinje content is determined by Northern blots using connexin 40- and 43-specific probes.

### EXAMPLE 2

### Cellular Engraftment to Generate Biological Pacemakers

A. Use of ES-Derived Conduction Cells to Generate a Biological Pacemaker.

A series of cardiac engraftment experiments demonstrate the usefulness of cellular grafts as a biological pacemaker. Cell populations enriched in node cells are selected as described in Example 1B, 1C or 1D and directly engrafted into the left ventricle of a syngeneic adult mouse. Cardiac automaticity is monitored throughout the experiment in vivo using surface electrocardiographic recordings. Three weeks post-engraftment, the heart is harvested and placed on a Langendorf/working heart apparatus. Functional hearts can be maintained in vitro for three to four hours on this apparatus. The anatomic origin of pacemaker activity is monitored by ECG leads placed on the epicardial surface. Heart block is induced pharmacologically (via perfusion) and/or surgically (via cautery) of the AV node. The capacity of the engrafted cells to drive ventricular depolarization is monitored through the epicardial ECG leads. Variables including optimal cell number to provide pacemaker activity (as determined by a resident βGAL reporter in the conduction cells) and anatomic position of the graft are ascertained in this system.

B. Use of Genetically Engineered Cells to Promote Neo-Innervation of a Biological Pacemaker.

A further series of experiments determines the extent to which expression of recombinant NGF within the graft promotes neo-innervation. It is known that myocardial grafts can be used to deliver recombinant protein to the myocardium, and that ectopic cardiac NGF expression leads to hyper-innervation in transgenic mice. An NGF expression construct is introduced into skeletal myoblasts (C2C12 cells). Stably transformed cell lines are established and their capacity to produce biologically active NGF is ascertained in an in vitro neurite outgrowth assay. To monitor the degree of neo-innervation, the genetically modified C2C12 cells are engrafted into an adult-syngeneic host. At three weeks post engraftment, the animals are sacrificed and the hearts harvested and processed for immunohistologic analyses. NGF fusion gene expression is monitored by in situ and anti-NGF immunohistologic analyses. The extent of neo-innervation is assessed using immunologic markers for both sympathetic and parasympathetic neurons, as well as by histologic examination and measurements of cardiac catecholamine content. Retrograde neuron labeling experiments are initiated with working heart preparations in vitro to track the anatomic origins of the nascent neurons within the heart. In all instances, myoblast grafts lacking the NGF transgene are employed as negative controls.

A further series of experiments is conducted in which the NGF-expressing cells are co-engrafted with selected pacemaker cells. These experiments determine the level at which NGF expression facilitates innervation of the pacemaker cells within the ectopic graft site. Once again, immunohistologic analyses determines the extent of neo-innervation. Negative controls include grafts lacking myoblasts, as well as grafts with myoblasts lacking the NGF transgene.

Additional experiments are initiated to determine the level of functional innervation. These experiments utilize retrograde labeling techniques to determine the extent to which the pacemaker graft is coupled to the cardiac ganglia. Subsequent physiologic experiments determine the extent to which the pacemaker graft is subject to autonomic regulation in instances where AV node activity is blocked pharmacologically and/or physically.

### EXAMPLE 3

### Generation of a Cellular Interface Between a Pacemaker Lead and the Host Myocardium

Traditional pacemakers activate ventricular depolarization through a bipolar lead which field stimulates a region of the endocardial surface in the apex of the ventricle. Because the electrical signal is dissipated through 360°, and because there is frequently fibrosis between the lead and the excitable myocardium, a significant amount of energy is needed to trigger cardiac depolarization. In the present experiment, a cellular interface is constructed between the pacemaker lead wire and the excitable myocardium. This would consist of an insulated wire which terminates with a collagen gel plug impregnated with cardiomyocytes, for example fetal cardiomyocytes obtained from differentiating ES cells as described herein and/or in WO 95/14079. The tip of this lead is inserted into the myocardium, facilitating coupling between the cardiomyocytes and the excitable myocardium. Because the pacemaker discharge does have to traverse a region of fibrosis (or has to traverse only a smaller region), and because the discharge will not be dissipated throughout a 360° area, stimulation of the interface ES-derived cardiomyocytes is accomplished with much lower energy utilization. Excitation of the host myocardium is actuated through nascent junctional complexes formed between the host and interface cardiomyocytes.

### EXAMPLE 4

### Restorative Cellular Engraftment

In this Example it is demonstrated that cellular engraftment may be used to restore function to injured regions of myocardium. Sonomicrometer crystals were placed 1.5 cm apart on the myocardial wall of an anesthetized CXMD (dystrophic) dog in an open chest procedure. Base line contractility measurements were recorded. Mongrel fetal cardiomyocytes, prepared by retrograde collagenase perfusion on a Langendorf apparatus, were then injected directly into the myocardium in between the sonomicrometer crystals. After a three-week recovery period, base line contractility was re-evaluated. Additionally, contractility in response to dobutamine challenge (at 20 μg/kg/min and 40 μg/kg/min) was also recorded.

Assessment of hemodynamic parameters indicated that the hearts were globally responding to dobutamine challenge as indicated (see Table 1 below).

TABLE 1

| | | Baseline | 20 μg/kg/min | 40 μg/kg/min |
|---|---|---|---|---|
| Heart Rate | Beats/min | 140 ± 17 | 179 ± 43* | 209 ± 35#* |
| LV systolic | mmHg | 97 ± 18 | 132 ± 23* | 114 ± 18# [symbol] |
| Pressure | | | | |
| Rate- | MmHG · Beats · | 13,637 ± | 22,822 ± | 23,941 ± |
| pressure | min⁻¹ | 3,264 | 7,997 | 6,439 |
| Product | | | | |

Repeated measure ANOVA followed by Student Newman-Keuls test.
Data are expressed as the mean ± one standard deviation.
#p < 0.001 vs. baseline.
*p < 0.01 vs. dobutamine 20 μg/kg/min [symbol]
p < 0.001 vs. dobutamine 20 μg/kg/min

To determine the effect of dobutamine on contractility, segment length shortening was normalized to pre-dobutamine value for each sonomicrometer pair according to the following formula:

Δ%SS=[(post dobutamine SS−pre-dobutamine SS)/pre-dobutamine SS]×100.

The study compared control (no injection), sham grafts (saline bolus) and cellular grafts (cellular bolus). In the control and the cellular graft groups, about a 5% increase in contractility (as measured as Δ%SS) was observed. In contrast, in the sham operated animals, which received a bolus saline injection and thus were subjected to regional injury due to needle damage, no increase in Δ%SS was observed. In the case of the 40 μg/kg/min perfusion experiment, this difference was statistically significant. It was thus evidenced cellular engraftment can be used to restore function to injured regions of myocardium.

### EXAMPLE 5

### Construction of a Cardiomyocyte-Seeded Filament

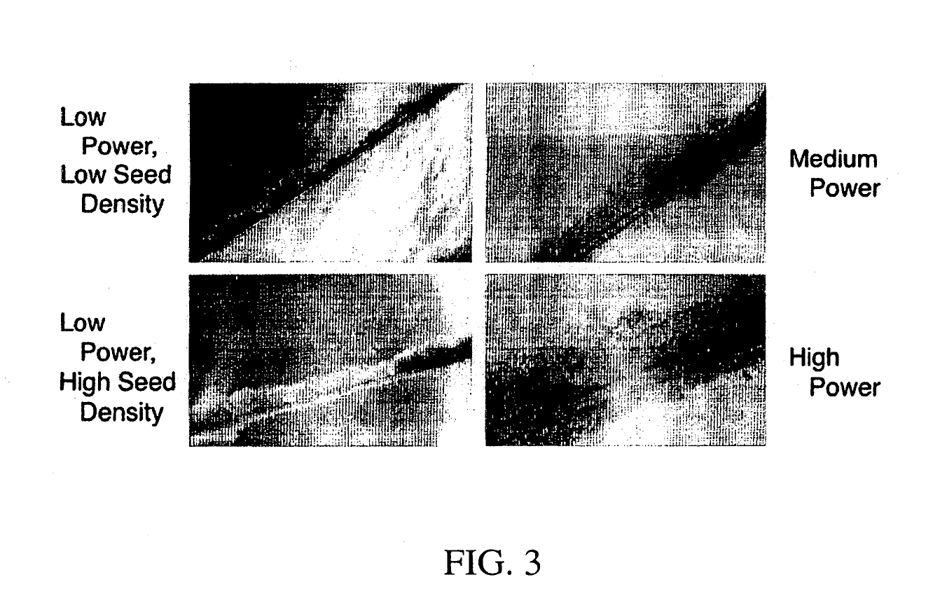

In studies with fetal canine cardiomyocytes, sutures were suspended between two metal bars attached to a petri dish, at approximately 5 mm above the bottom of the plate. A dispersed cell preparation of fetal cardiomyocytes was then plated into the dish such that the suture was submerged. The cultures were then placed in an incubator, where the dishes were subjected to mild agitation. 24 hrs later, the media was changed and the non-adherent cells were removed. Fetal cardiomyocytes were observed to have seeded upon the sutures. After three days of culture, rhythmic contractions were observed on the seeded sutures. FIG. 3 depicts illustrative ones of these seeded sutures.

### EXAMPLE 6

### Illustrative Uses of a Cardiomyocyte-Seeded Filaments

Once ES- or fetal-derived Purkinje cells are in hand, we will generate an “artificial Purkinje fiber”. A series of experiments will be performed to optimize the seeding procedure. This will include a number of straight forward experiments which will test the timing of seeding post differentiation, the effects of varying the concentration of cell, optimization of the suture material (i.e. collagen coated, fibronectin coated, etc.), and optimization of culture agitation (rotating vs. rocking, rate, etc.). The experimental endpoints consist of measuring the percentage of the suture which is successfully seeded, as well as determine the cell thickness on the suture. The capacity of the suture to propagate action potentials is also tested. In first pass screens this is easily accomplished by simply monitoring contraction of the cells along the suture. In addition, quantitative monitoring of the action potential propagation is performed using voltage sensitive dyes. Microelectrode records are also performed. If necessary, cells are placed at the edge of the structure with an extracellular electrode and monitor action potential propagation.

Once seeded sutures are generated which are able to propagate action potentials, they are used in experiments designed to synchronize contraction between primary cardiomyocyte cultures in adjacent chambers on a multichamber slide. Towards that end, the seeded suture are draped across the dam dividing the two chambers, and primary cardiomyocytes are seeded in each chamber. The dam is modified (lowered) so as to allow the suture to be completely immersed in culture media. After the cells are attached and exhibit automaticity, cells in one chamber are paced (using extracellular electrodes) at a rate in excess of the spontaneous rate of the cultures. Extracellular electrode records are obtained simultaneously in cells in the non-paced chamber. If the seeded suture is able to propagate the action potential, cells in the non-paced chamber should contract at a rate identical to those in the paced chamber.

The establishment of successful generation of seeded sutures suitable to drive pacing in vitro will lead to experiments to determine if cells present on the seeded suture are viable in vivo. Towards that end, a short segment of seeded suture is affixed to the surface of a syngeneic adult mouse heart. The ends of the suture are inserted into the epicardial surface (by “burying” the end into a small hole produced with a 30 gauge syringe needle) The central region of the suture is allowed to lie freely on the epicardial surface of the heart. Three weeks post engraftment, the heart is removed and the viability of the cells on the suture is monitored by histologic analyses. Any inflammatory responses to the presence of the sutures is monitored by standard histochemistry (H and E, Masson's trichrome) and immune histology (anti-macrophage anti-leukocyte, etc.).

Once viable suture implants are obtained, their use for pacing applications is evaluated. Towards that end a short seeded suture is affixed between the left atrium and left ventricle of a mouse heart. After recovery, complete A-V block is induced by treatment with anti-RHO antibodies as described by El Sherif and colleagues. (Serum and immunoglobulin G from the mother of a child with congenital heart block induce conduction abnormalities and inhibit L-type calcium channels in a rat heart model. Pediatric Research. July 1998. 44: 11-19, 1998.) The animals are then intubated, the chest opened, and the right atrium paced with a stimulating electrode at a rate greater than the spontaneous rate of the ventricle. Synchronous pacing of the ventricle in these animals (but not in controls lacking seeded sutures, nor in controls with non-seeded sutures) will indicate that the seeded suture has electrically coupled the left atrium and ventricle. Similar experiments can also be conducted in the dog.

All publications cited herein are indicative of the level of skill in the art and are hereby incorporated by reference in their entirety as if each had been individually incorporated by reference and fully set forth.

What is claimed is:

1. A cellular population comprising cardiomyocytes obtained by differentiation of a multipotent cell and which is enriched in conduction cardiomyocytes relative to working cardiomyocytes.

2. The cellular population of claim 1, wherein at least 50% of said cardiomyocytes in said cellular population are conduction cardiomyocytes.

3. The cellular population of claim 1, wherein the conduction cardiomyocytes comprise sino-atrial node cells.

4. The cellular population of claim 1, wherein the conduction cardiomyocytes comprise atrial-ventricular node cells.

5. The cellular population of claim 1, wherein the conduction cardiomyocytes comprise sino-atrial and atrial-ventricular node cells.

6. The cellular population of claim 5, wherein the conduction cardiomyocytes also comprise Perkinje cells.

7. A method for obtaining a cellular population enriched in conduction cardiomyocytes, comprising:
providing a transgenic multipotent cell carrying a conduction cardiomyocyte selection marker enabling selection of conduction cardiomyocytes from other cells;
causing said multipotent cell to differentiate to form said conduction cardiomyocytes and other cells; and
selecting said conduction cardiomyocytes based on said selection marker.

8. The method of claim 7, wherein said conduction cardiomyocyte selection marker comprises a positive selection gene fused to a promoter which causes expression of the gene in said conduction cardiomyocytes but not in said other cells.

9. The method of claim 8, wherein the positive selection gene is an antibiotic resistance gene, and said selecting includes contacting said conduction cardiomyocytes and other cells with an antibiotic to kill said other cells but not said conduction cardiomyocytes.

10. The method of claim 9, wherein the promoter is selected from the group consisting of the minK promoter and the connexin40 promoter.

11. The method of claim 9, wherein said promoter is the connexin40 promoter.

12. The method of claim 9, wherein said promoter is the minK promoter.

13. The method of claim 7, wherein said conduction cardiomyocyte selection marker comprises a cardiomyocyte selection marker is a negative selection gene fused to a promoter which causes expression of the gene in said other cells but not in said conduction cardiomyocytes, and said selecting includes contacting said conduction cardiomyocytes and other cells with a negative selection agent to kill said other cells but not said conduction cardiomyocytes.

14. The method of claim 13, wherein said negative selection gene is the HSV thymidine kinase gene, and said selecting includes contacting said conduction cardiomyocytes and other cells with gancyclovir to kill said other cells but not said conduction cardiomyocytes.

15. The method of claim 14, wherein said promoter is the connexin43 promoter.

16. The method of claim 13, which comprises:
providing said multipotent cell having a cardiomyocyte selection marker which enables selection of a general cardiomyocyte cell population including said conduction cardiomyocytes and other cardiomyocytes, and a conduction cardiomyocyte selection marker which enables selection of said conduction cardiomyocytes from the other cardiomyocytes;
causing said multipotent cells to differentiate to form a mixed cell population including cardiomyocytes;
first selecting the general cardiomyocyte cell population from said mixed population based on the general cardiomyocyte selection marker; and
second selecting a conduction cardiomyocyte population from the general cardiomyocyte cell population based on said conduction cardiomyocyte selection marker.

17. The method of claim 16, wherein said general cardiomyocyte selection marker comprises an antibiotic resistance gene fused to a first promoter which causes expression of the gene specifically in cardiomyocytes, and wherein said first selecting includes contacting the mixed cell population with an antibiotic agent.

18. The method of claim 17, wherein said conduction cardiomyocyte selection marker is a negative selection gene fused to a second promoter which causes expression of the gene in cardiomyocytes other than said conduction cardiomyocytes, but not in said conduction cardiomyocytes, and wherein said second selecting includes contacting the general cardiomyocyte cell population with a negative selection agent.

19. The method of claim 18, wherein said negative selection gene is the HSV thymidine kinase gene, and wherein said second selecting includes contacting the general cardiomyocyte cell population with gancyclovir.

20. The method of claim 19, wherein said second promoter is the connexin43 promoter.

21. A transgenic multipotent cell having DNA encoding a selection gene fused to a promoter, said DNA enabling selection of a cell population enriched in conduction cardiomyocytes relative to working cardiomyocytes.

22. The cell of claim 21, wherein said selection gene is an antibiotic resistance gene, and said promoter causes expression of the gene specifically in said conduction cells or enhancedly in said conduction cells relative to said working cardiomyocytes.

23. The cell of claim 22, wherein said promoter is the minK promoter.

24. The cell of claim 22, wherein said promoter is the connexin40 promoter.

25. The cell of claim 21, wherein said selection gene is a negative selection gene, and said promoter causes expression of said gene in said working cardiomyocytes but not in said conduction cardiomyocytes.

26. The cell of claim 25, wherein said negative selection gene is the HSV thymidine kinase gene.

27. The cell of claim 25, wherein said promoter is the connexin43 promoter.

28. The cell of claim 21, wherein said DNA comprises:
a general cardiomyocyte cell selection marker which enables selection of an essentially pure cardiomyocyte cell population differentiated from said stem cell, said essentially pure cardiomyocyte cell population including conduction and working cardiomyocytes; and
a conduction cardiomyocyte selection marker which enables selection of a population from said essentially pure cardiomyocyte cell population which is enriched in conduction cells.

29. The cell of claim 28, wherein said general cardiomyocyte cell selection marker includes an antibiotic resistance gene fused to a first promoter which causes expression of the gene specifically in cardiomyocytes.

30. The cell of claim 29, wherein said conduction cardiomyocyte selection marker is a negative selection gene fused to a second promoter which causes expression of the gene in the working cardiomyocytes but not in the conduction cardiomyocytes.

31. The cell of claim 30, wherein said negative selection gene is the HSV thymidine kinase gene.

32. The cell of claim 31, wherein said second promoter is the connexin43 promoter.

33. A vector useful for transformation of a multipotent cell, the vector comprising DNA including a selection gene fused to a promoter, said DNA enabling selection of a cell population enriched in conduction cardiomyocytes relative to working cardiomyocytes.

34. The vector of claim 33, wherein said selection gene is an antibiotic resistance gene, and said promoter causes expression of said gene in said conduction cardiomyocytes but not in said working cardiomyocytes.

35. The vector of claim 34, wherein said promoter is the minK promoter.

36. The vector of claim 35, wherein said promoter is the connexin40 promoter.

37. The vector of claim 34, wherein said promoter is the minK promoter.

38. The vector of claim 40, wherein said selection gene is a negative selection gene fused to a promoter which causes expression of said gene in said working cardiomyocytes but not in said conduction cardiomyocytes.

39. The vector of claim 38, wherein said negative selection gene is the HSV thymidine kinase gene.

40. The vector of claim 39, wherein said promoter is the connexin43 promoter.

41. A method for pacing a heart of a mammal, comprising:
providing a cellular population containing fetal node cardiomyocytes or embryonic node cardiomyocytes; and
introducing said cellular population into myocardial tissue of said mammal, wherein said node cardiomyocytes pace said heart.

42. The method of claim 41, wherein said node cardiomyocytes include sino-atrial node cells.

43. The method of claim 41, wherein said node cardiomyocytes include atrial-ventricular node cells.

44. The method of claim 41, wherein said node cardiomyocytes include sino-atrial and atrial-ventricular node cells.

45. The method of claim 41, wherein said cellular population also includes Perkinje cells.

46. A device for implantation into the myocardium of a mammal, comprising:
a substrate adapted for in vivo implantation into the myocardium; and
viable cardiomyocytes coated on said substrate.

47. The device of claim 46, wherein:
said substrate is a conductive lead.

48. The device of claim 47, comprising:
a carrier matrix on said conductive lead; and viable cardiomyocytes in said carrier matrix.

49. The device of claim 46, wherein said cardiomyocytes are fetal cardiomyocytes.

50. The device of claim 49 wherein said carrier matrix is a gel.

51. The device of claim 50 wherein said gel is a collagen gel.

52. A method for cellular engraftment in injured myocardial tissue of the heart of a mammal, comprising:
implanting cells into myocardial tissue of a mammal, said myocardial tissue having suffered injury; and
said implanting conducted during active granulation and tissue formation following the injury.

53. The method of claim 52, wherein said cells are cardiomyocytes.

54. A method for cellular engraftment in injured myocardial tissue of the heart of a mammal, comprising:
implanting cells into injured myocardial tissue of a mammal; and
said implanting conducted so as to provide a region of engrafted cells, said region contacting uninjured myocardial tissue and extending into said injured myocardial tissue.

55. The method of claim 54, wherein said cells are cardiomyocytes.

56. A cellular population comprising an in vitro derived population of cardiomyocytes, said population of cardiomyocytes being enriched in conduction cardiomyocytes relative to working cardiomyocytes.

57. The cellular population of claim 56, wherein at least 50% of said cardiomyocytes in said cellular population are conduction cardiomyocytes.

58. The cellular population of claim 56, wherein the conduction cardiomyocytes comprise sino-atrial node cells.

59. The cellular population of claim 56, wherein the conduction cardiomyocytes comprise atrial-ventricular node cells.

60. The cellular population of claim 56, wherein the conduction cardiomyocytes comprise sino-atrial and atrial-ventricular node cells.

61. The cellular population of claim 60, wherein the conduction cardiomyocytes also comprise Perkinje cells.
